(19) United States
(12) Patent Application Publication
(10) Pub. No.: US 2007/0162037 A1
(43) Pub. Date: Jul. 12, 2007

(54) Apparatus for displacing knee prosthesis

(75) Inventors: Ing-Ho Chen, Hualien City (TW); Shou-Heng Pao, Hsinchu (TW)

Correspondence Address: TROXELL LAW OFFICE PLLC, SUITE 1404, 5205 LEESBURG, FALLS CHURCH, VA, 22041, US

(73) Assignees: Ing-Ho Chen; United Orthopedic Corporation

(21) Appl. No.: 11/315,484

(22) Filed: Dec. 23, 2005

(51) Int. Cl. A61F 5/00

(52) U.S. Cl. 606/87

(57) ABSTRACT

An apparatus for displacing knee prosthesis comprises a cutting auxiliary member having two hollow posts at one side thereof and a plurality of grooves thereon forming at a predetermined angle, a measuring member having pegs on one side thereof, wing parts extending outwardly from middle of the measuring member, wherein a plurality of adjustment holes is arranged at predetermined angle on the wing parts, a measuring unit being movably equipped at a top of the measuring member, and a position member including a fitting board and two fixing plates respectively extending from a bottom of the fitting board, wherein the fitting board has scale holes at two ends thereof being matchable with the adjustment holes. Thereby, the apparatus is used for measuring an appropriate and more precise size for cutting femur bones before displacing knee prosthesis and to minimize a surgical wound.

### BACKGROUND OF THE INVENTION

(a) Field of the Invention

The present invention relates to an apparatus for displacing knee prosthesis, and particularly to an apparatus for measuring an appropriate and more precise size for cutting femur bones before displacing knee prosthesis and to minimize a surgical wound.

(b) Description of the Prior Art

In general, prior to displacing knee prosthesis, a conventional measuring apparatus shown in FIG. 11 is used for cutting femur bones comprising: a base 5 having an extension board 51, a measuring portion 52 including cutting type panel 521 and turning angle part 522, and a view window 53 for viewing the turning angle part 522. A part of the cutting type panel 521 is exposed to an edge of the base 5. The measuring portion 52 further forming a press plate 54 at an end thereof. A concavity 55 is defined in a side of the base 5 for accommodating an extrusive element 56 which cooperates with the measuring portion 52 to move up and down in the concavity 55.

Although the conventional apparatus can be used for displacing knew prosthesis, the base 5 and measuring portion 52 are assembled together for use and that shape into large area; accordingly, to set the conventional apparatus on a femur for measuring users have to make a long cut of more than twenty centimeter. Such a long cut results in a broad wound and not only make patients getting hurt also need more time to regain health.

### SUMMARY OF THE INVENTION

Accordingly, an object of the present invention is to provide an apparatus for displacing knee prosthesis and for measuring an appropriate and more precise size of cuts on femur bones before displacing knee prosthesis and simultaneously minimizing a surgical wound.

To achieve the above object, an apparatus for displacing knee prosthesis includes a cutting auxiliary member having two hollow posts at one side thereof and a plurality of grooves thereon forming at a predetermined angle, a measuring member having pegs on one side thereof, wing parts extending outwardly from middle of the measuring member, wherein a plurality of adjustment holes is arranged at predetermined angle on the wing parts, a measuring unit being movably equipped at a top of the measuring member, and a position member including a fitting board and two fixing plates respectively extending from a bottom of the fitting board, wherein the fitting board has scale holes at two ends thereof being matchable with the adjustment holes.

Other objects, advantages and novel features of the present invention will be drawn from the following detailed embodiment of the present invention with the drawings.

### BRIEF DESCRIPTION OF THE DRAWINGS

FIG. 1 and FIG. 2 are perspective views of a cutting auxiliary member of an apparatus for displacing knee prosthesis of the present invention.

FIG. 3 and FIG. 4 are perspective views of a measuring member of the apparatus.

FIG. 5 and FIG. 6 are perspective views of a position member of the apparatus.

FIG. 7 to FIG. 10 are schematic operation views of the apparatus; and is an enlarged, sectional and operational view of the vacuum breaker in FIG. 3.

FIG. 11 is a schematic operation view of prior art.

### DETAILED DESCRIPTION OF THE PREFERRED EMBODIMENTS

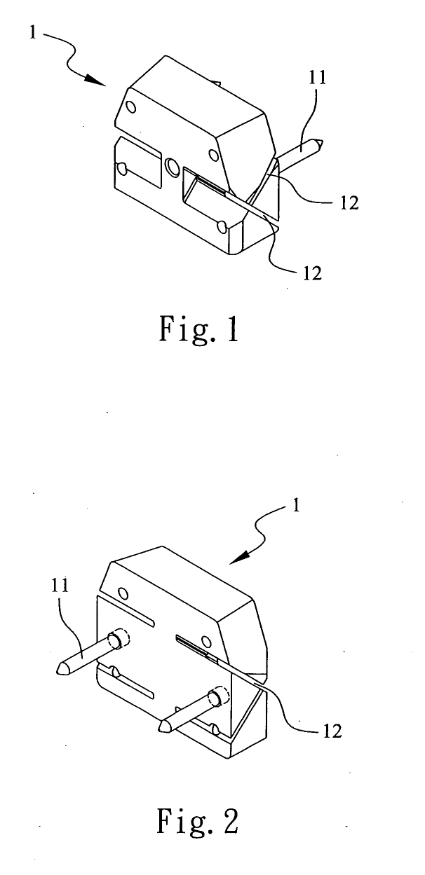

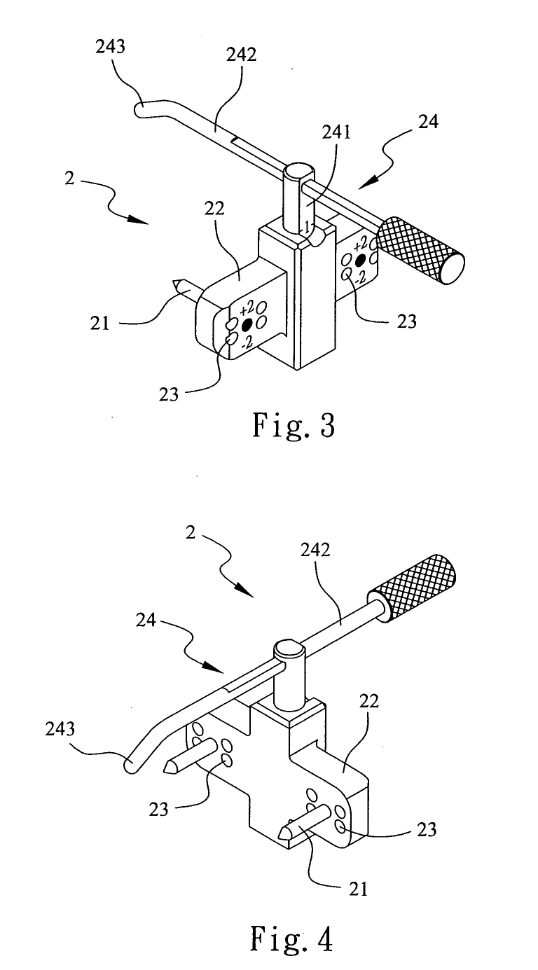

Referring to FIGS. 1 to FIG. 6, an apparatus for displacing knee prosthesis comprises a cutting auxiliary member 1, a measuring member 2 and a position member 3, which is used for measuring an appropriate and more precise size of cuts on femur bones before displacing knee prosthesis and simultaneously minimizing a surgical wound.

The cutting auxiliary member 1 has at least two hollow posts 11 at one side thereof and a plurality of grooves 12 thereon forming at a predetermined angle.

The measuring member 2 has pegs 21 on one side thereof, wing parts 22 extending outwardly from middle of the measuring member 2, wherein a plurality of adjustment holes 23 is arranged at predetermined angle on the wing parts, and a measuring unit 24 being movably equipped at a top of the measuring member 2. The measuring unit 24 includes a ruler 241 which is movably disposed at a top of the measuring member 2 and an adjustment rod 242 which is connected with an end of the ruler 241, wherein the adjustment rod 242 forms a bending portion 243 at one end thereof.

The position member 3 including a fitting board 31 and two fixing plates 32 respectively extending from a bottom of the fitting board 31, wherein the fitting board 31 has scale holes 33 at two ends thereof being matchable with the adjustment holes 23.

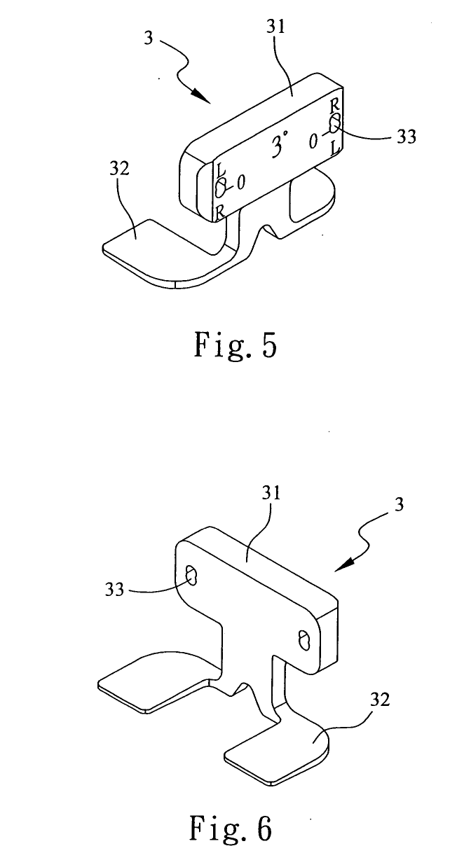

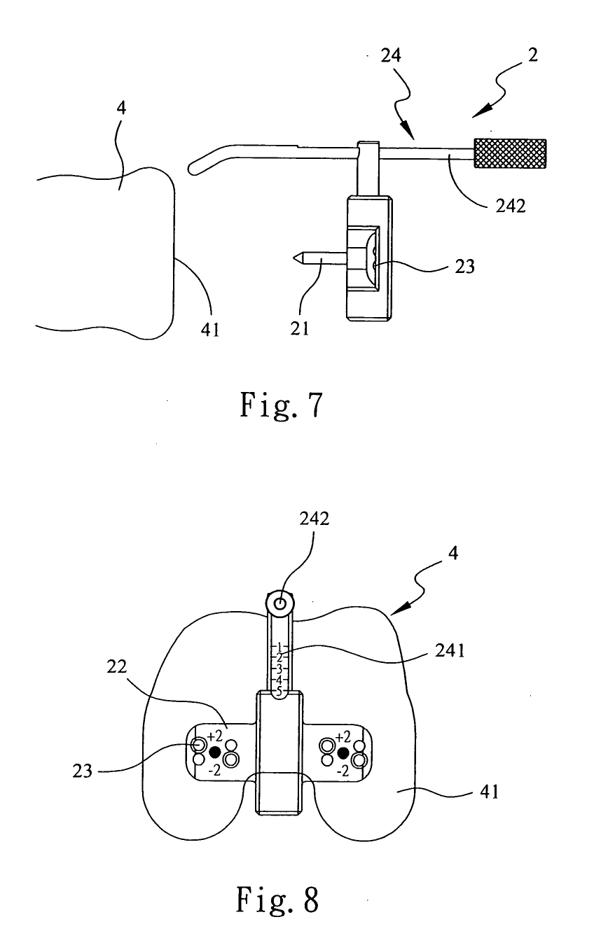

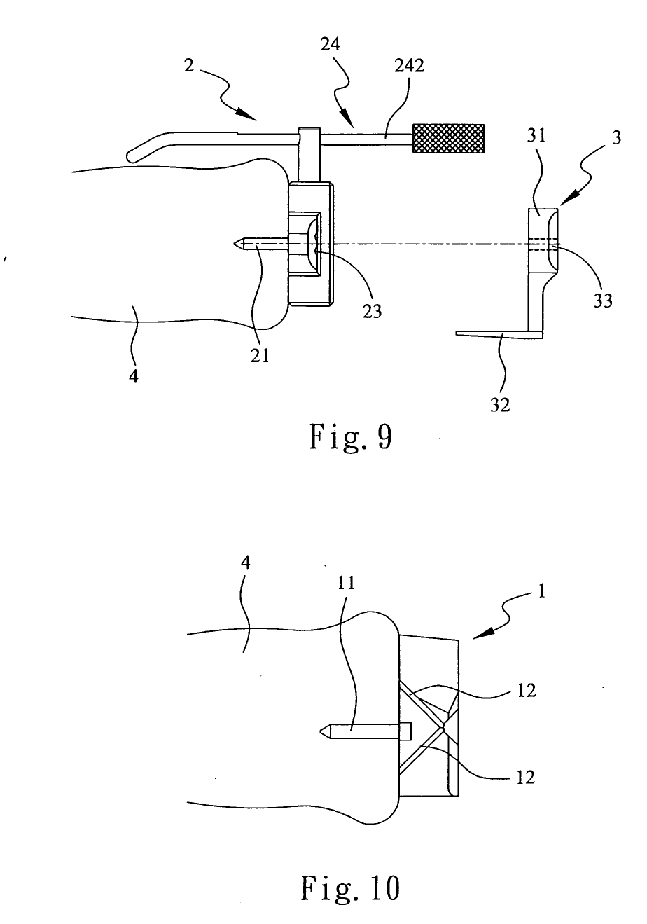

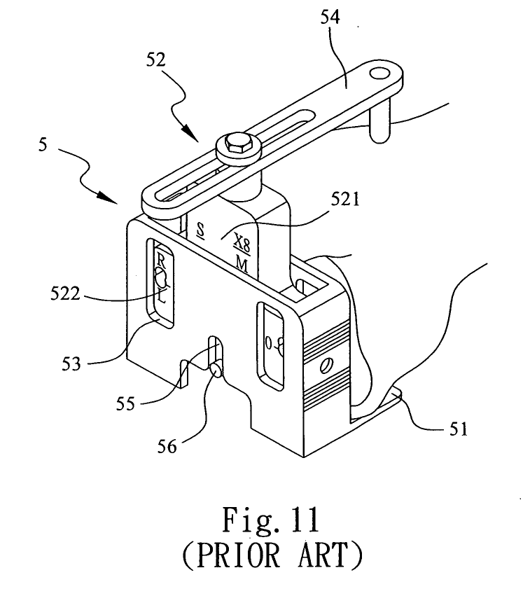

Referring to FIGS. 7-10, prior to performing a knee prosthesis replacement surgery, a surgeon has to cut a patient's femur 4 with an appropriate size and angle. To do so, first, cut the patient's knee to form an opening then cut an end of the femur 4 as a plane surface 41, set the measuring member 2 on the plane surface 41 by inserting the pegs 21 into the plane surface 41 (shown as FIG. 7), and manipulate the measuring unit 24 (shown as FIG. 5) of the measuring member 2 to get a correct model of the cutting auxiliary member 1. When measuring, pull out the adjustment rod 242 and lay down the bending portion 243 on a horizontal face of the patient's femur 4, thereby the ruler 241 simultaneously indicates a correct model number of the cutting auxiliary member 1; secondly, put the fixing plate 32 of the position member 3 against a bottom the femur 4 and to match the scale hole 33 of the fitting board 31 with the adjustment holes 23 to show up an appropriate outward turning angle (shown as FIG. 9) for cutting the femur 4, and then put off the measuring member 2 and the position member 3; finally, apply the correct cutting auxiliary member 1 to the plan surface 41 by inserting the two hollow posts 11 into holes drilled by the pegs 21 before; accordingly, the surgeon can cut the femur 4 from the groove 12 of the cutting auxiliary member 1 (shown as FIG. 10) with a right size and an appropriate angle.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. An apparatus for displacing knee prosthesis comprising:
a cutting auxiliary member having at least two hollow posts at one side thereof and a plurality of grooves thereon forming at a predetermined angle;
a measuring member having pegs on one side thereof, wing parts extending outwardly from middle of the measuring member, wherein a plurality of adjustment holes is arranged at predetermined angle on the wing parts, and a measuring unit being movably equipped at a top of the measuring member; and
a position member including a fitting board and two fixing plates respectively extending from a bottom of the fitting board, wherein the fitting board has scale holes at two ends thereof being matchable with the adjustment holes.

2. The apparatus for displacing knee prosthesis of claim 1, wherein the measuring unit has a ruler being movably disposed at a top of the measuring member and an adjustment rod being connected with an end of the ruler.

3. The apparatus for displacing knee prosthesis of claim 2, wherein the ruler marks numbers thereon for showing models of the cutting auxiliary member.

4. The apparatus for displacing knee prosthesis of claim 3, wherein the adjustment rod forms a bending portion at one end thereof.
